(19) United States
(12) Patent Application Publication
(10) Pub. No.: US 2026/0159376 A1
(43) Pub. Date: Jun. 11, 2026

(54) SENSOR

(71) Applicant: KABUSHIKI KAISHA TOSHIBA, Minato-ku, Tokyo (JP)

(72) Inventors: Yosuke AKIMOTO, Yokohama Kanagawa (JP); Shihe WANG, Kawasaki Kanagawa (JP); Hiroshi HAMASAKI, Hiratsuka Kanagawa (JP); Yui YAMAZAKI, Ota Tokyo (JP)

(73) Assignee: KABUSHIKI KAISHA TOSHIBA, Minato-ku, Tokyo (JP)

(21) Appl. No.: 19/257,594

(22) Filed: Jul. 2, 2025

(30) Foreign Application Priority Data
Dec. 9, 2024 (JP) 2024-214168

(51) Int. Cl. B81B 3/00; G01N 33/00

(52) U.S. Cl. CPC B81B 3/0086; G01N 33/004; B81B 2201/0214; B81B 2203/04; B81B 2207/07

(57) ABSTRACT

According to one embodiment, a sensor includes a base, a first detection portion, and a controller. The first detection portion includes a first base electrode fixed to the base, a first fixed portion fixed to the base, and a first element supported by the first fixed portion. The first element includes first and second conductive members. A gap is provided between the first base electrode and the first element. The controller performs first and second operations. In the first operation, the controller is configured to detect a first capacitance signal corresponding to a first capacitance between the first base electrode and a one conductive member of the first conductive member and the second conductive member. In the second operation, the controller is configured to detect a first electrical resistance of an other conductive member of the first conductive member and the second conductive member.

### CROSS-REFERENCE TO RELATED APPLICATIONS

This application is based upon and claims the benefit of priority from Japanese Patent Application No. 2024-214168, filed on Dec. 9, 2024; the entire contents of which are incorporated herein by reference.

### FIELD

Embodiments described herein relate generally to a sensor.

### BACKGROUND

For example, there is a sensor using a MEMS (Micro Electro Mechanical Systems) element, etc. It is desirable to improve the characteristics of the sensor.

### BRIEF DESCRIPTION OF THE DRAWINGS

FIG. 1 is a schematic cross-sectional view illustrating a sensor according to a first embodiment;

FIG. 2 is a schematic plan view illustrating the sensor according to the first embodiment;

FIG. 3 is a schematic plan view illustrating the sensor according to the first embodiment;

FIG. 4 is a schematic plan view illustrating the sensor according to the first embodiment;

FIG. 5 is a schematic cross-sectional view illustrating a part of the sensor according to the first embodiment; and

FIG. 6 is a schematic cross-sectional view illustrating a sensor according to the first embodiment.

### DETAILED DESCRIPTION

According to one embodiment, a sensor includes a base, a first detection portion, and a controller. The first detection portion includes a first base electrode fixed to the base, a first fixed portion fixed to the base, and a first element supported by the first fixed portion. The first element includes a first conductive member and a second conductive member. A first gap is provided between the first base electrode and the first element. The controller is configured to perform a first operation and a second operation. In the first operation, the controller is configured to detect a first capacitance signal corresponding to a first capacitance between the first base electrode and a one conductive member of the first conductive member and the second conductive member. In the second operation, the controller is configured to detect a first electrical resistance of an other conductive member of the first conductive member and the second conductive member.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously or illustrated in an antecedent drawing are marked with like reference numerals, and a detailed description is omitted as appropriate.

### First Embodiment

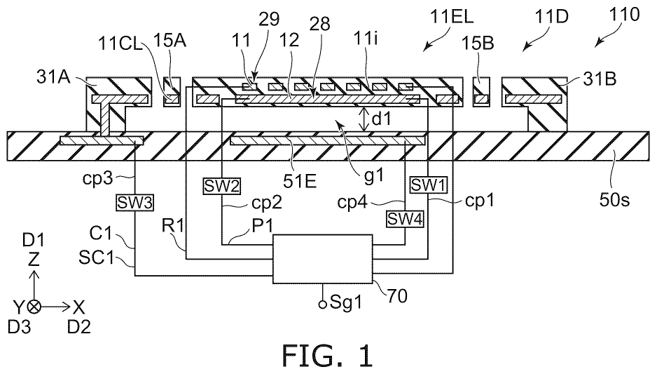

FIG. 1 is a schematic cross-sectional view illustrating a sensor according to the first embodiment.

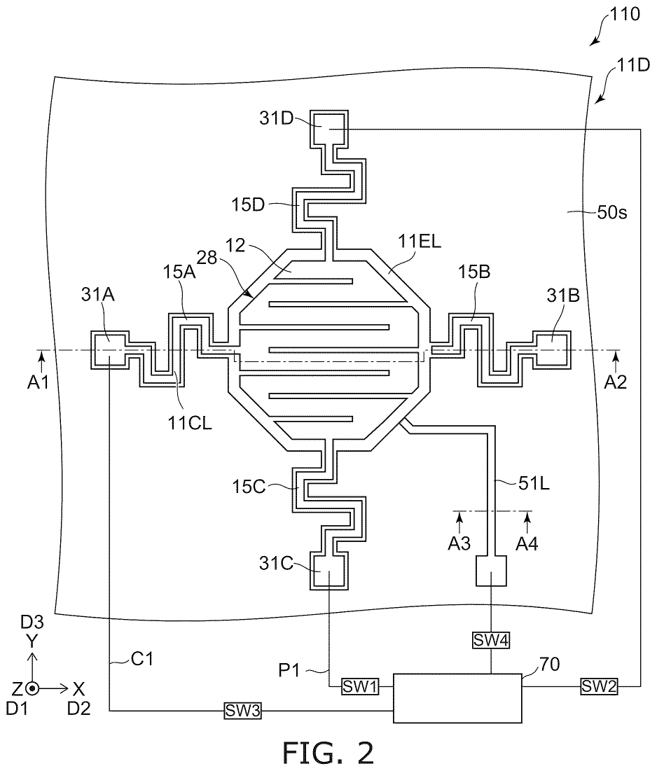

FIGS. 2 to 4 are schematic plan views illustrating the sensor according to the first embodiment.

FIG. 1 is a cross-sectional view taken along the line A1-A2 in FIGS. 2 to 4.

As shown in FIG. 1, a sensor 110 according to the embodiment includes a base 50s, a first detection portion 11D, and a controller 70.

The first detection portion 11D includes a first base electrode 51E fixed to the base 50s, a first fixed portion 31A fixed to the base 50s, and a first element 11EL supported by the first fixed portion 31A. The first element 11EL includes a first conductive member 11 and a second conductive member 12. A first gap g1 is provided between the first base electrode 51E and the first element 11EL.

The controller 70 is configured to perform a first operation and a second operation. In the first operation, the controller 70 is configured to detect a first capacitance signal SC1 corresponding to a first capacitance C1 between the first base electrode 51E and a one conductive member 28 of the first conductive member 11 and the second conductive member 12. In one example, the one conductive member 28 of the first conductive member 11 and the second conductive member 12 may be the second conductive member 12.

In the second operation, the controller 70 is configured to detect a first electrical resistance R1 of an other conductive member 29 of the first conductive member 11 and the second conductive member 12. In a case where the one conductive member 28 of the first conductive member 11 and the second conductive member 12 is the second conductive member 12, the other conductive member 29 of other one of the first conductive member 11 and the second conductive member 12 is the first conductive member 11. The first conductive member 11 and the second conductive member 12 are interchangeable. The following mainly describes the case where the one conductive member 28 is the second conductive member 12 and the other conductive member 29 is the first conductive member 11.

As described above, in the embodiment, in the first operation, the controller 70 detects the first capacitance signal SC1 corresponding to the first capacitance C1. In the second operation, the controller 70 detects the first electrical resistance R1. Thus, two types of detection results are obtained. The detection target may be detected based on these detection results.

For example, the first electrical resistance R1 may be configured to change according to the detection target around the first element 11EL. For example, the thermal characteristics (e.g., heat dissipation) of the first element 11EL change depending on the state of the detection target. This is due to the thermal conduction characteristics of the detection target. The first electrical resistance R1 of the other conductive member 29 (first conductive member 11) changes due to the change in the thermal characteristics of the first element 11EL. By detecting the change in the first electrical resistance R1, the detection target (e.g., gas) can be detected. The detection target may be, for example, a gas such as carbon dioxide. The sensor 110 functions as, for example, a gas sensor. A value based on the first electrical resistance R1 may correspond to the detection result of the detection target.

On the other hand, a distance d1 between the first base electrode 51E and the one conductive member 28 (second conductive member 12) may change depending on the detection target. For example, the distance d1 may change due to a change in a connecting member, which will be described later, depending on the detection target. The change in the first capacitance signal SC1 corresponding to the first capacitance C1 based on the change in distance d1 may be detected. The detection target may be detected by detecting the change in the first capacitance signal SC1.

In the embodiment, a plurality of types of detection results are obtained. It is possible to provide a sensor whose characteristics can be improved.

Furthermore, for example, one of the plurality of detection results may be used to correct the other of the plurality of detection results. In one example, the distance d1 may change due to temperature change or change over time. When the distance d1 changes, the heat dissipation characteristics of the heat of the first element 11EL through the first gap g1 change. The change in the heat dissipation characteristics due to the change in the distance d1 adversely affects the change in the first electrical resistance R1 according to the detection target. By compensating for the effect due to the change in the distance d1, the detection target can be accurately detected using the first electrical resistance R1.

As shown in FIG. 1, the controller 70 may be further configured to perform a third operation to output an output signal Sg1. The output signal Sg1 is obtained by correcting a value based on the first electrical resistance R1 based on the first capacitance signal SC1. For example, the correction is performed based on information about the relationship between the first capacitance signal SC1 and the distance d1; and the relationship between the distance d1 and the first electrical resistance R1. This allows for more accurate detection results to be obtained. In the third operation, for example, calibration is performed. The controller 70 may include, for example, a processor.

In the embodiment, a coefficient relating to the relationship between the first electrical resistance R1 of the other conductive member 29 of the first conductive member 11 and the second conductive member 12, and the concentration of the detection target may be corrected based on the detection result of the capacitance. For example, the detection result of the first electrical resistance R1 is converted to the concentration of the detection target (e.g., gas concentration) based on the coefficient. The converted value (concentration) corresponds to the value based on the first electrical resistance R1. In this case as well, the detection target can be accurately detected by correcting the coefficient based on the detection result of the capacitance.

The controller 70 may be configured to output information corresponding to the distance d1 between the first base electrode 51E and the one conductive member 28 (the second conductive member 12).

In the second operation, the controller 70 may be configured to supply a first power P1 to the one conductive member 28 (second conductive member 12). The first power P1 increases the temperature of the first element 11EL, and in accordance with this, the temperature of the other conductive member 29 (first conductive member 11) is increased. The temperature of the other conductive member 29 (first conductive member 11) has a value that corresponds to the state of the detection object. The detection object can be detected by detecting the first electrical resistance R1 that corresponds to the temperature of the other conductive member 29 (first conductive member 11).

On the other hand, in the first operation, the controller 70 may not supply the first power P1 to the one conductive member 28 (the second conductive member 12). As described above, in the first operation, the first capacitance signal SC1 corresponding to the first capacitance C1 is detected. In such a first operation, the first power P1 may not be supplied.

In this example, the one conductive member 28 (the second conductive member 12) functions as a heater or a capacitance electrode. By applying plurality of functions, it is possible to improve characteristics with a simple structure.

The above-mentioned plurality of operations may be switched by a switch or the like. As shown in FIG. 1, the sensor 110 may include a first switch SW1 and a second switch SW2. The first switch SW1 is provided in a first current path cp1 between a part of the one conductive member 28 (second conductive member 12) and the controller 70. The second switch SW2 is provided in a second current path cp2 between the other part of the one conductive member 28 (second conductive member 12) and the controller 70. The first switch SW1 and the second switch SW2 are in a non-conductive state in the first operation. The first switch SW1 and the second switch SW2 are in a conductive state in the second operation. By such switch operation, the first power P1 is not supplied in the first operation, and the first power P1 is supplied in the second operation.

As shown in FIG. 1, the sensor 110 may further include a third switch SW3 and a fourth switch SW4. The third switch SW3 is provided in a third current path cp3 between the one conductive member 28 (second conductive member 12) and the controller 70. In this example, the one conductive member 28 (second conductive member 12) is electrically connected to the third switch SW3 via a first element wiring layer 11CL described later. The fourth switch SW4 is provided in a fourth current path cp4 between the first base electrode 51E and the controller 70. The third switch SW3 and the fourth switch SW4 are in the conductive state in the first operation. The third switch SW3 and the fourth switch SW4 are in the non-conductive state in the second operation. By the operation of such switches, the first capacitance signal SC1 corresponding to the first capacitance C1 is detected in the first operation.

As shown in FIG. 1, in this example, the one conductive member 28 (second conductive member 12) is located between the first base electrode 51E and the other conductive member 29 (first conductive member 11) in a first direction D1 from the first base electrode 51E to the first element 11EL.

The first direction D1 is defined as a Z-axis direction. One direction perpendicular to the Z-axis direction is defined as an X-axis direction. A direction perpendicular to the Z-axis and X-axis directions is defined as a Y-axis direction.

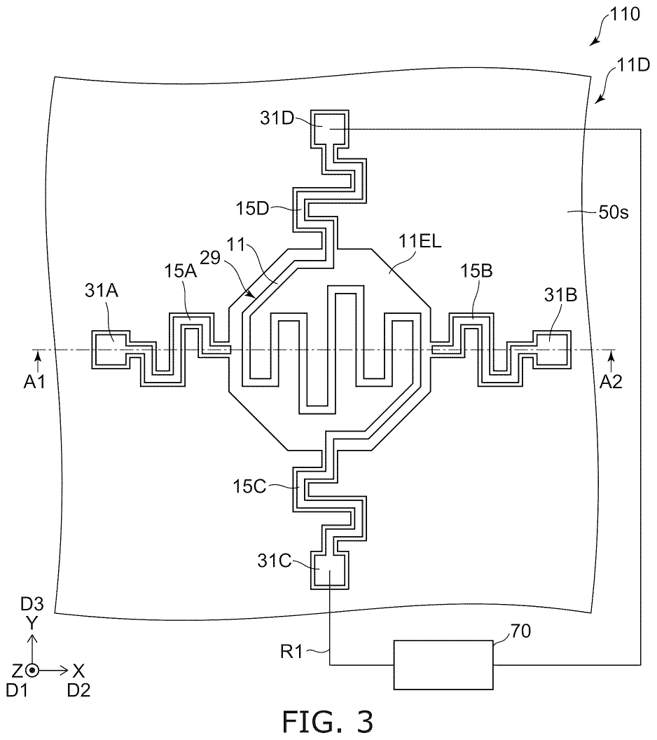

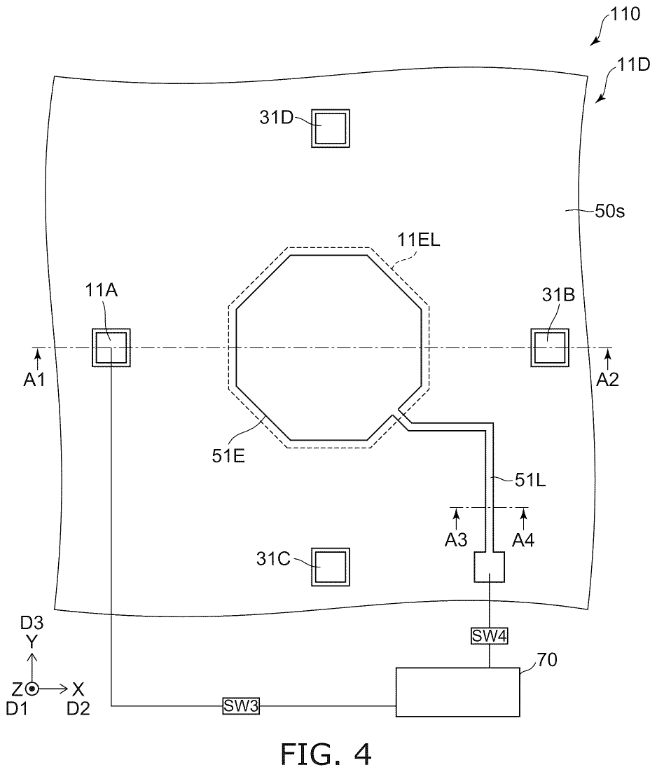

FIG. 2 illustrates an example of a pattern of the one conductive member 28 (second conductive member 12). FIG. 3 illustrates an example of a pattern of the other conductive member 29 (first conductive member 11). FIG. 4 illustrates an example of a pattern of the first base electrode 51E. As shown in these figures, the first element 11EL is a layer along the X-Y plane.

As shown in FIGS. 1 and 2, the first detection portion 11D may further include a first connecting member 15A. The first connecting member 15A is supported by the first fixed portion 31A. The first connecting member 15A supports the first element 11EL. A part of the first gap g1 is provided between the base 50s and the first connecting member 15A.

As shown in FIG. 2, a length of the first connecting member 15A along an extension direction is longer than a width along a crossing direction crossing the extension direction of the first connecting member 15A. The first connecting member 15A may have, for example, a meandering structure. The first connecting member 15A may have, for example, a spring structure. For example, the conduction of heat from the first element 11EL through the connecting member can be suppressed.

As shown in FIG. 1 and FIG. 2, the first detection portion 11D may further include a second fixed portion 31B fixed to the base 50s and a second connecting member 15B supported by the second fixed portion 31B. The second connecting member 15B supports the first element 11EL. A direction from the first connecting member 15A to the second connecting member 15B (for example, the second direction D2) crosses the first direction D1. The second direction D2 may be, for example, the X-axis direction.

As shown in FIG. 2, the first detection portion 11D may include a third fixed portion 31C, a third connecting member 15C, a fourth fixed portion 31D, and a fourth connecting member 15D. The third fixed portion 31C is fixed to the base 50s. The third connecting member 15C is supported by the third fixed portion 31C. The fourth fixed portion 31D is fixed to the base 50s. The fourth connecting member 15D is supported by the fourth fixed portion 31D. The third connecting member 15C supports the first element 11EL. The fourth connecting member 15D supports the first element 11EL. A direction from the third connecting member 15C to the fourth connecting member 15D (e.g., the third direction D3) crosses the direction from the first connecting member 15A to the second connecting member 15B (the second direction D2). The third direction D3 crosses a plane including the first direction D1 and the second direction D2, for example.

By using the plurality of connecting members described above, the first element 11EL is stably supported.

As shown in FIGS. 1 and 2, the first detection portion 11D may include a first element wiring layer 11CL. The first element wiring layer 11CL is electrically connected to the one conductive member 28 (second conductive member 12). The first element wiring layer 11CL passes through, for example, the first connecting member 15A.

As shown in FIG. 2, in this example, the conductive layer electrically connected to a part of the one conductive member 28 (second conductive member 12) passes through the third connecting member 15C. The conductive layer electrically connected to the other part of the one conductive member 28 passes through the fourth connecting member 15D.

As shown in FIG. 3, in this example, the conductive layer electrically connected to a part of the other conductive member 29 (first conductive member 11) passes through the third connecting member 15C. The conductive layer electrically connected to the other part of the other conductive member 29 passes through the fourth connecting member 15D.

A position in the Z-axis direction of the conductive layer electrically connected to the one conductive member 28 (second conductive member 12) is different from a position in the Z-axis direction of the conductive layer electrically connected to the other conductive member 29 (first conductive member 11).

In the embodiment, the conductive layer electrically connected to the one conductive member 28 or the other conductive member 29 may pass through any of the first connecting member 15A, the second connecting member 15B, the third connecting member 15C, and the fourth connecting member 15D.

As shown in FIG. 1, the first element 11EL may include an insulating member 11i. At least a part of the insulating member 11i is provided between the one conductive member 28 (second conductive member 12) and the other conductive member 29 (first conductive member 11).

As shown in FIG. 4, the first detection portion 11D may include a first base wiring layer 51L. The first base wiring layer 51L is electrically connected to the first base electrode 51E. It is preferable that at least a part of the first base wiring layer 51L does not overlap the first connecting member 15A in the first direction D1. It is preferable that at least a part of the first base wiring layer 51L does not overlap the above-mentioned connecting member in the first direction D1. The interaction between the first base wiring layer 51L and the connecting member is suppressed. For example, the effect of unevenness caused by the first base wiring layer 51L on the characteristics of the connecting member is suppressed. Below, an example of unevenness caused by the first base wiring layer 51L is described.

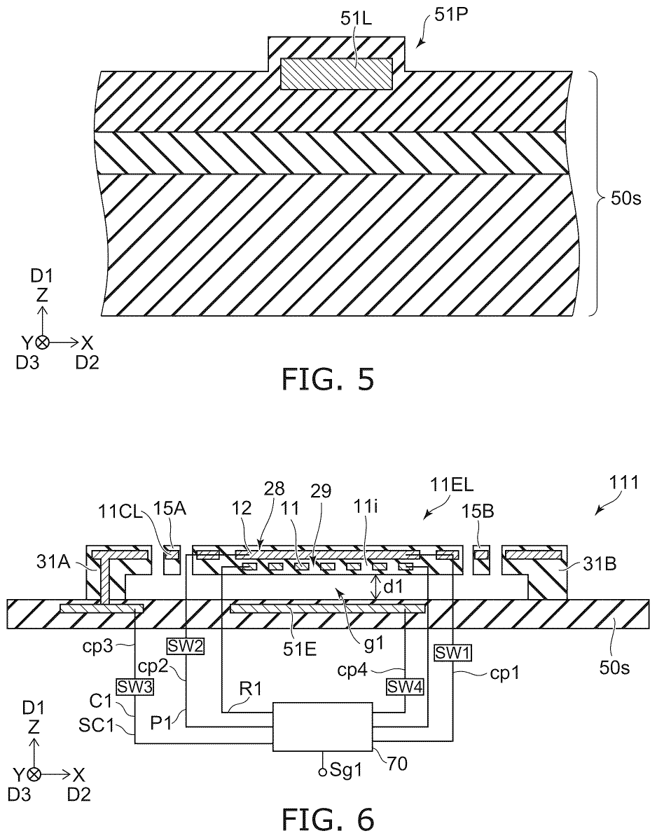

FIG. 5 is a schematic cross-sectional view illustrating a part of the sensor according to the first embodiment.

FIG. 5 is a cross-sectional view taken along the line A3-A4 in FIGS. 2 and 4. As shown in FIG. 5, the first base wiring layer 51L is provided on the base 50s. A protruding portion 51P is formed by the first base wiring layer 51L. When such a protruding portion 51P overlaps any of a part of the plurality of connecting members, the thermal characteristics (heat dissipation, etc.) of the connecting member change partially. By the first base wiring layer 51L not overlapping the connecting members, it becomes easier to obtain uniform characteristics.

FIG. 6 is a schematic cross-sectional view illustrating a sensor according to the first embodiment.

FIG. 6 is a cross-sectional view corresponding to the line A1-A2 in FIGS. 2-4. As shown in FIG. 6, in a sensor 111 according to the embodiment, the positional relationship between the two conductive members differs from that in the sensor 110. The configuration of sensor 111 except for this may be the same as the configuration of sensor 110.

In the sensor 111, the other conductive member 29 (first conductive member 11) is located between the first base electrode 51E and the one conductive member 28 (second conductive member 12) in the first direction D1 from the first base electrode 51E to the first element 11EL.

In this case as well, the first switch SW1 is provided in the first current path cp1 between a part of the one conductive member 28 (second conductive member 12) and the controller 70. The second switch SW2 is provided in the second current path cp2 between the other part of the one conductive member 28 (second conductive member 12) and the controller 70. The first switch SW1 and the second switch SW2 are in the non-conductive state in the first operation. The first switch SW1 and the second switch SW2 are in the conductive state in the second operation.

The third switch SW3 is provided in the third current path cp3 between the one conductive member 28 (second conductive member 12) and the controller 70. In this example, the one conductive member 28 (second conductive member 12) is electrically connected to the third switch SW3 via the first element wiring layer 11CL. The fourth switch SW4 is provided in the fourth current path cp4 between the first base electrode 51E and the controller 70. The third switch SW3 and the fourth switch SW4 are in the conductive state in the first operation. The third switch SW3 and the fourth switch SW4 are in the non-conductive state in the second operation.

In this example, the other conductive member 29 (first conductive member 11) functions as a temperature detection resistor or a capacitance electrode. By applying plurality of functions, it is possible to improve characteristics with a simple structure.

In the embodiment, the controller 70 may be configured to repeatedly perform a set including the first operation and the second operation. The controller 70 may be configured to repeatedly perform a set including the first operation, the second operation, and the third operation. For example, periodic calibration is performed to track.

The embodiment may include the following Technical proposals:

### Technical Proposal 1

A sensor comprising:
a base;
a first detection portion; and
a controller,
the first detection portion including:
   a first base electrode fixed to the base,
   a first fixed portion fixed to the base, and
   a first element supported by the first fixed portion,
the first element including a first conductive member and a second conductive member,
a first gap being provided between the first base electrode and the first element,
the controller being configured to perform a first operation and a second operation,
in the first operation, the controller being configured to detect a first capacitance signal corresponding to a first capacitance between the first base electrode and a one conductive member of the first conductive member and the second conductive member, and
in the second operation, the controller being configured to detect a first electrical resistance of an other conductive member of the first conductive member and the second conductive member.

### Technical Proposal 2

The sensor according to Technical proposal 1, wherein
the controller is further configured to perform a third operation to output an output signal, and
the output signal is obtained by correcting a value based on the first electrical resistance based on the first capacitance signal.

### Technical Proposal 3

The sensor according to Technical proposal 1 or 2, wherein
the controller is configured to supply a first power to the one conductive member in the second operation.

### Technical Proposal 4

The sensor according to Technical proposal 3, wherein
the controller does not supply the first power to the one conductive member in the first operation.

### Technical Proposal 5

The sensor according to any one of Technical proposals 1-4, wherein
the one conductive member is between the first base electrode and the other conductive member in a first direction from the first base electrode to the first element.

### Technical Proposal 6

The sensor according to any one of Technical proposals 1-4, wherein
the other conductive member is between the first base electrode and the one conductive member in a first direction from the first base electrode to the first element.

### Technical Proposal 7

The sensor according to Technical proposal 5 or 6, wherein
the first detection portion further includes a first connecting member,
the first connecting member is supported by the first fixed portion, and
the first connecting member supports the first element.

### Technical Proposal 8

The sensor according to Technical proposal 7, wherein
the first detection portion further includes a first base wiring layer electrically connected to the first base electrode, and
at least a part of the first base wiring layer does not overlap the first connecting member in the first direction.

### Technical Proposal 9

The sensor according to Technical proposal 8, wherein
the first base wiring layer is provided on the base, and
a protruding portion is formed by the first base wiring layer.

### Technical Proposal 10

The sensor according to any one of Technical proposals 7-9, wherein
the first detection portion further includes a first element wiring layer electrically connected to the one conductive member, and
the first element wiring layer passes through the first connecting member.

### Technical Proposal 11

The sensor according to any one of Technical proposals 7-10, wherein
a part of the first gap is between the base and the first connecting member, and
a length of the first connecting member along an extending direction is longer than a width of the first connecting member along a crossing direction crossing the extending direction.

### Technical Proposal 12

The sensor according to any one of Technical proposals 7-11, wherein
the first detection portion further includes:
   a second fixed portion fixed to the base, and
   a second connecting member supported by the second fixed portion,
the second connecting member supports the first element, and
a direction from the first connecting member to the second connecting member crosses the first direction.

### Technical Proposal 13

The sensor according to Technical proposal 12, wherein
the first detection portion further includes:
   a third fixed portion fixed to the base,
   a third connecting member supported by the third fixed portion,
   a fourth fixed portion fixed to the base, and
   a fourth connecting member supported by the fourth fixed portion,
the third connecting member supports the first element,
the fourth connecting member supports the first element, and
a direction from the third connecting member to the fourth connecting member crosses a direction from the first connecting member to the second connecting member.

### Technical Proposal 14

The sensor according to Technical proposal 13, wherein
a conductive layer electrically connected to the one conductive member or the other conductive member passes through any one of the first connecting member, the second connecting member, the third connecting member, and the fourth connecting member.

### Technical Proposal 15

The sensor according to any one of Technical proposals 1-14, further comprising:
a first switch; and
a second switch,
the first switch being provided in a first current path between a part of the one conductive member and the controller,
the second switch being provided in a second current path between another part of the one conductive member and the controller,
the first switch and the second switch are in a non-conductive state in the first operation,
the first switch and the second switch are in a conductive state in the second operation.

### Technical Proposal 16

The sensor according to any one of Technical proposals 1-15, further comprising:
a third switch; and
a fourth switch,
the third switch being provided in a third current path between the one conductive member and the controller,
the fourth switch is provided in a fourth current path between the first base electrode and the controller,
the third switch and the fourth switch are in a conductive state in the first operation, and
the third switch and the fourth switch are in a non-conductive state in the second operation.

### Technical Proposal 17

The sensor according to any one of technical proposals 1-16, wherein
the controller is configured to output information corresponding to a distance between the first base electrode and the one of the conductive members.

### Technical Proposal 18

The sensor according to Technical proposal 1, wherein
the controller is configured to repeatedly perform a set of the first operation and the second operation.

### Technical Proposal 19

The sensor according to Technical proposal 2, wherein
the controller is configured to repeatedly perform a set of the first operation, the second operation, and the third operation.

### Technical Proposal 20

The sensor according to any one of technical proposals 1-19, wherein
the first electrical resistance is configured to change in response to a detection target around the first element.

According to the embodiment, a sensor capable of improving characteristics can be provided.

In the specification, “electrically connected” includes a state in which plurality of conductors are physically in contact with each other and current flows between these plurality of conductors. “Electrically connected” includes a state in which a conductor is inserted between plurality of conductors and current flows between these plurality of conductors.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as bases, detection portions, controllers, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors practicable by an appropriate design modification by one skilled in the art based on the sensors described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor comprising:
a base;
a first detection portion; and
a controller,
the first detection portion including:
a first base electrode fixed to the base,
a first fixed portion fixed to the base, and
a first element supported by the first fixed portion,
the first element including a first conductive member and a second conductive member,
a first gap being provided between the first base electrode and the first element,
the controller being configured to perform a first operation and a second operation,
in the first operation, the controller being configured to detect a first capacitance signal corresponding to a first capacitance between the first base electrode and a one conductive member of the first conductive member and the second conductive member, and
in the second operation, the controller being configured to detect a first electrical resistance of an other conductive member of the first conductive member and the second conductive member.

2. The sensor according to claim 1, wherein
the controller is further configured to perform a third operation to output an output signal, and
the output signal is obtained by correcting a value based on the first electrical resistance based on the first capacitance signal.

3. The sensor according to claim 1, wherein
the controller is configured to supply a first power to the one conductive member in the second operation.

4. The sensor according to claim 3, wherein
the controller does not supply the first power to the one conductive member in the first operation.

5. The sensor according to claim 1, wherein
the one conductive member is between the first base electrode and the other conductive member in a first direction from the first base electrode to the first element.

6. The sensor according to claim 1, wherein
the other conductive member is between the first base electrode and the one conductive member in a first direction from the first base electrode to the first element.

7. The sensor according to claim 5, wherein
the first detection portion further includes a first connecting member,
the first connecting member is supported by the first fixed portion, and
the first connecting member supports the first element.

8. The sensor according to claim 7, wherein
the first detection portion further includes a first base wiring layer electrically connected to the first base electrode, and
at least a part of the first base wiring layer does not overlap the first connecting member in the first direction.

9. The sensor according to claim 8, wherein
the first base wiring layer is provided on the base, and
a protruding portion is formed by the first base wiring layer.

10. The sensor according to claim 7, wherein
the first detection portion further includes a first element wiring layer electrically connected to the one conductive member, and
the first element wiring layer passes through the first connecting member.

11. The sensor according to claim 7, wherein
a part of the first gap is between the base and the first connecting member, and
a length of the first connecting member along an extending direction is longer than a width of the first connecting member along a crossing direction crossing the extending direction.

12. The sensor according to claim 7, wherein
the first detection portion further includes:
a second fixed portion fixed to the base, and
a second connecting member supported by the second fixed portion,
the second connecting member supports the first element, and
a direction from the first connecting member to the second connecting member crosses the first direction.

13. The sensor according to claim 12, wherein
the first detection portion further includes:
a third fixed portion fixed to the base,
a third connecting member supported by the third fixed portion,
a fourth fixed portion fixed to the base, and
a fourth connecting member supported by the fourth fixed portion,
the third connecting member supports the first element,
the fourth connecting member supports the first element, and
a direction from the third connecting member to the fourth connecting member crosses a direction from the first connecting member to the second connecting member.

14. The sensor according to claim 13, wherein
a conductive layer electrically connected to the one conductive member or the other conductive member passes through any one of the first connecting member, the second connecting member, the third connecting member, and the fourth connecting member.

15. The sensor according to claim 1, further comprising:
a first switch; and
a second switch,
the first switch being provided in a first current path between a part of the one conductive member and the controller,
the second switch being provided in a second current path between another part of the one conductive member and the controller,
the first switch and the second switch are in a non-conductive state in the first operation,
the first switch and the second switch are in a conductive state in the second operation.

16. The sensor according to claim 1, further comprising:
a third switch; and
a fourth switch,
the third switch being provided in a third current path between the one conductive member and the controller,
the fourth switch is provided in a fourth current path between the first base electrode and the controller,
the third switch and the fourth switch are in a conductive state in the first operation, and
the third switch and the fourth switch are in a non-conductive state in the second operation.

17. The sensor according to claim 1, wherein
the controller is configured to output information corresponding to a distance between the first base electrode and the one of the conductive members.

18. The sensor according to claim 1, wherein
the controller is configured to repeatedly perform a set of the first operation and the second operation.

19. The sensor according to claim 2, wherein
the controller is configured to repeatedly perform a set of the first operation, the second operation, and the third operation.

20. The sensor according to claim 1, wherein
the first electrical resistance is configured to change in response to a detection target around the first element.
